(19) United States
(12) Patent Application Publication
(10) Pub. No.: US 2011/0223255 A1
(43) Pub. Date: Sep. 15, 2011

(54) IMPLANTABLE PRODUCTS COMPRISING NANOPARTICLES

(75) Inventors: Burghard Thiesen, Berlin (DE); Andreas Jordan, Berlin (DE)

(73) Assignee: MAGFORCE NANOTECHNOLOGIES AG, Berlin (DE)

(21) Appl. No.: 12/864,793

(22) PCT Filed: Feb. 11, 2009
(86) PCT No.: PCT/DE2009/000196

§ 371 Date: Dec. 1, 2010

Related U.S. Application Data
(60) Provisional application No. 61/071,084, filed on Apr. 11, 2008.

(30) Foreign Application Priority Data
Feb. 11, 2008 (DE) 10 2008 008 522.7

(51) Int. Cl. A61K 9/14; A61K 33/26; A61K 31/395; A61K 31/337; A61K 38/16; A61K 31/407; A61K 31/704; A61P 35/00; B82Y 5/00

(52) U.S. Cl. 424/489; 424/400; 424/647; 514/183; 514/449; 514/19.3; 514/410; 514/34; 977/773

(57) ABSTRACT

The present disclosure relates to nanoparticle-containing implantable and preferably biodegradable medical products and their use for the thermotherapeutic after-treatment after surgical removal of tumors and cancerous ulcers.

### BACKGROUND OF THE INVENTION

1. Field of the Invention

The present invention relates to implantable products containing nanoparticles and their use in medicine, particularly for thermotherapeutic after-treatment after surgical removal of tumors and cancerous ulcers.

2. Description of the Relevant Art

After surgical removal of tumor tissue, the problem arises almost always that tumor cells will still remain inside the body (incomplete resection). Following closure of the wound, these tumor cells may be able to grow up again to larger tumors and/or to metastases. For this reason, chemotherapeutic after-treatments are carried out which are very stressful to the patient. However, as a minimum of healthy tissue should be removed, the operating surgeon must compromise on a, preferably, complete tumor resection and removal of a minimum of healthy tissue.

### SUMMARY OF THE INVENTION

One embodiment is directed to a solid or gel-like medical product heatable by an alternating magnetic field, wherein the medical product is provided in the form of a physiologically acceptable tissue, sponge, film or gel and wherein magnetic particles are contained in the medical product which will generate heat if excited by an alternating magnetic field and thereby will heat the medical product. The particles may be stationary embedded into or attached to the medical product. In an embodiment, the particles will remain permanently in the medical product, will not be released by diffusion and will only be released in the case of biodegradable medical products due to the degradation process. In an embodiment, the particles may be microparticles or nanoparticles. The particles may be paramagnetic or superparamagnetic.

In an embodiment, the medical product is deformable and can be adjusted to the surface contours of a tissue or organ or the field of surgery after surgical tumor removal. The medical product may be biodegradable. The medical product may be bioresorbable within one to twelve months. In an embodiment, the medical product is impregnated, coated or soaked with the magnetic particles. The medical product may be physiologically tolerated and may be degraded to physiologically tolerated components. In an embodiment, the medical product is flexible and does not consist of a metal or a metal alloy and wherein the medical product is not provided in form of an aqueous solution of the particles.

The biodegradable medical product releases the magnetic particles and/or delivers them to surrounding tumor tissue or tumor cells. The magnetic particles may be provided at concentrations of 100 micrograms to 2 grams per gram of the medical product. In an embodiment, the medical product further comprises at least one therapeutic effective agent. The therapeutic agent may be bound adhesively, ionically, covalently or via a linker to the particle. Detachment of the therapeutic agent, in an embodiment, is initiated by an alternating magnetic field.

### DETAILED DESCRIPTION OF THE PREFERRED EMBODIMENTS

Described herein are products and methods for a more effective after-treatment after cancer surgery.

Surprisingly, it was found that implantable medical products containing nanoparticles heatable in alternating magnetic fields will enable a significantly improved after-treatment of cancer surgery in comparison to chemotherapy if these medical products are implanted or placed into the surgery area.

Therefore, embodiments relate to a solid or gel-like medical product heatable in alternating magnetic fields in which the medical product is present in the form of a physiologically acceptable tissue, sponge, film or gel, wherein magnetic particles are contained in the medical product that generate heat when excited by an alternating magnetic field and thus heating the medical product.

It is crucial for the inventive medical product that the particles, i.e. the particles excitable by an alternating magnetic field, are embedded stationary into or adhere to the medical product.

Conventionally, aqueous solutions of magnetic particles are produced to either direct the particles loaded with pharmacologic drugs to a specific target location through a static magnetic field or aqueous solutions of particles excitable in an alternating magnetic field are injected directly into a tumor in order for the particles to accumulate in the tumor cells and to destroy the tumor cells by heat generation. The heat is generated in first place by the loss of hysteresis heat of the particles.

The inventive medical products are not aqueous or physiological aqueous solutions or suspensions of the magnetic particles but solid or gel-like carriers such as a tissue or a film in which the particles are embedded stationary. Provided that it is not about biodegradable medical products the particles will permanently remain within the medical product and the medical product will permanently remain at the implant position, similar to a dental implant or an artificial knee joint.

As the particles will remain permanently in the medical product, will not be eluted by diffusion and will only be released by a degradation process in the case of biodegradable medical products, the area the implanted medical product is positioned at can still be heated after any desired period of time, i.e. one week after implantation, one month after implantation, one year after implantation, as well as ten years after implantation.

Preferred embodiments relate to biodegradable medical products which are degradable at variable rates by human and animal bodies depending on the indication. However, the particles are not released from these medical products by diffusion but within the limits of biological degradation only. Thus, dissolution will occur with this bioresorbable medical product in which the remaining remnants of the medical product undergoing dissolution can be heated further by an alternating magnetic field.

However, it is crucial for the inventive medical products that they are flexible or deformable and will be able to follow the surface contours of a tissue or an organ or the operative field after surgical tumor removal. Hence, the inventive medical products are in the form of tissues which can be placed onto tissues or organs or an operative field and will follow the uneven surfaces without a problem, or is in the form of a gel, a film-forming composition or a film-forming spray which by their nature can be applied to any uneven surface.

Herein, an operative field refers to the field that is delimited by the out-most edges of a surgical wound. In other words, the operative field is the transient region or the border region between tumor and healthy tissue. Treatment of after-treatment of this field is very important to prevent formation of recurrences.

The medical products described herein are applied to, coated onto the operative field and in case of a spray, sprayed onto the operative field and thus are destined for after-treatment of a surgical wound after tumor surgery.

Thus, the inventive medical products primarily are not intended for systemic application but for implantation in the operative field. As the inventive medical products should remain in the operative field preferably for the course of the ensuing chemotherapy, the inventive medical products are biodegradable according to the time frame of planned therapy sessions, are bioresorbable for a longer period of time or are non-degradable.

It is important that the inventive medical products, preferably biodegradable or slowly biodegradable medical products are not in rigid form but may adjust flexibly to the surface of the operative field to be covered.

Hence, flexible, easily malleable, easily adjustable to other forms or formless medical products or carriers for the heatable or warmable particles are preferred in particular.

Medical products according to certain embodiments are all non-rigid and non-metallic carriers which adjust to a given surface and cover it to the greatest extent and moreover are suitable for uptake of magnetic particles, in particular superparamagnetic nanoparticles. The preferably biodegradable inventive medical products are medical cellulose, bandaging materials, wound inserts, surgical suture material, compresses, sponges, medical textiles, ointments, gels or film-forming sprays.

The medical cellulose and the medical textiles constitute preferably two-dimensional structures of low thickness which are impregnated with the particles. The magnetic particles attach to the fiber structure of this medical product which after surgery is put into the wound at the area of surgery in dry or pre-wetted form.

Sponges or biodegradable porous three-dimensional structures in general, which can contain the magnetic particles on the surface as well as in the cavities inside the porous structure as well as in the spongy material itself, are another form of the inventive medical products. After surgery these sponges are placed into the wound and will fill the area of surgery to the greatest extent, or only partially. The magnetic particles can be released from these sponge-like structures, wherein the particles can be present in solidly bound form as well. The release can be affected by diffusion of only loosely bound particles from the cavities of the porous structure as well as by biodegradation of the spongy structure if the particles are incorporated or embedded into in the material of the spongy structure itself.

The inventive medical products are destined for implantation into the human and animal body and must be physiologically acceptable. It is important that the inventive medical products are not present in liquid form as a solution or suspension but as a formulation which is viscous or thick or film-forming or solid so that after implantation the medical product will surely remain at the desired position.

It is likewise important that the medical product adjusts to any surfaces, i.e. it follows the surface contours.

Herein, a carrier of the magnetic particles is designated as a “medical product”, and the tissues, celluloses, gels, film-forming compositions, etc. described herein in detail serve as “carriers” which can be biodegradable or biostable and are non-magnetic and thus are not heatable in an alternating magnetic field without the magnetic particles. The carriers are made out of non-living matter, may contain X-ray markers or contrast media and bind the particles preferably adhesively and/or covalently. The particles, however, are mostly non-biodegradable, will release heat by excitation in an alternating magnetic field and thus will not only heat themselves but also the carrier, that's to say the whole medical product, too, and thereby the surrounding tissue as well. Moreover, pharmacological drugs such as cytostatics may be incorporated into the medical product as described below which can be released by diffusion and/or biodegradation of the carrier and/or heat generation and/or the alternating magnetic field to combat tumor cells first and foremost.

Herein, any medically usable textile or cellulose is labeled “tissue” from which bandaging materials, wound inserts, bandages or other medical clothes or fabrics are produced.

The phrase “biodegradable medical product” relates explicitly to the matrix of the magnetic particles only but not to the magnetic particles themselves which usually are non-biodegradable. Therefore, the medical cellulose, bandaging materials, wound inserts, surgical suture material, compresses, sponges, medical textiles, ointments, gels or film-forming sprays are biodegradable wherein or whereon the magnetic particles are applied to or incorporated into. Hence, the matrix for the magnetic particles of the degradable medical products with magnetic particles is biodegraded, i.e. the medical product without the magnetic particles, and the magnetic particles will usually remain or accumulate in the tumor tissue or cancer cells, respectively, and are mostly not biodegraded or a part of their coating only is biodegraded wherein the magnetic core is usually not biodegradable.

The area where the removed tumor or the removed cancer tissue was present is defined as the operative field.

Another preferred alternative of inventive medical products are liquid or gel-like formulations in the form of ointments, creams, gels and sprays, particularly film-forming sprays. These formulations contain the magnetic particles and will be applied to or sprayed onto the area of surgery after removal of the tumor.

With the exception of the magnetic particles, the inventive medical products are preferably biodegradable and therefore will dissolve completely preferably within one to twelve months, more preferably one to six months, wherein also the contained magnetic particles are released.

The functional principle of the inventive medical products is that they should cover the operative field as complete as possible in order that the magnetic particles will come as close as possible to the still remaining cancer cells or still remaining cancer tissue. The magnetic particles and preferably superparamagnetic particles can be heated in an alternating magnetic field wherein the still remaining cancer cells will be killed by thermotherapy. Herein the magnetic particles contained in the inventive medical product will heat the medical product as a whole and the magnetic particle diffused out of the medical product will heat the cancer cells to which they will adhere or which they will penetrate.

Moreover, the thermotherapeutic treatment can support conventional chemotherapy or radiotherapy because thermotherapeutic treatment will cause comparably few adverse effects and can be performed with a chemotherapeutic treatment at the same time. As the inventive medical products should cover the operative field or should fill out the area of surgery as complete as possible, respectively, the inventive medical products are in preferably direct contact with the still remaining cancer cells and the still remaining cancer tissue which can be killed in a particularly effective manner by the immediate proximity of the magnetic particles. The thermotherapeutic treatment with the inventive medical products is therefore considerably more selective and sparing than chemotherapy and radiotherapy.

In one embodiment at least one pharmacologically active compound, preferably an anti-cancer drug, is bound to said magnetic particles. Examples for suitable anti-cancer drugs are: actinomycin, aminoglutethimide, amsacrin, anastrozol, antagonists of purine or pyrimidine bases, anthracycline, aromatase inhibitors, asparaginase, anti-estrogens, bexaroten, bleomycin, buserelin, busulfan, camptothecin derivatives, capecitabin, carboplatin, carmustin, chlorambucil, cisplatin, cladribine, cyclophosphamide, cytarabine (cytosinarabinosid), alkylating cytostatics, dacarbazine, dactinomycin, daunorubicin, docetaxel, doxorubicin (adriamycin), epirubicin, estramustin, etoposide, exemestan, fludarabine, fluorouracil, folic acid antagonists, formestan, gemcitabine, glucocorticoids, goselerin, hormones and hormone antagonists, hycamtin, hydroxyurea, idarubicin, ifosfamide, imatinib, irinotecan, letrozol, leuprorelin, lomustin, melphalan, mercapto-purine, methotrexate, miltefosin, mitomycine, mitosis inhibitors, mitoxantrone, nimustine, oxaliplatin, paclitaxel, pentostatin, procarbazine, tamoxifen, temozolomide, teniposide, testolacton, thiotepa, tioguanine, topoisomerase inhibitors, topotecan, treosulfan, tretinoin, triptorelin, trofosfamide, vinblastin, vincristin, vindesin, vinorelbin, cytostatically effective antibiotics.

The detachment of at least one therapeutically active drug from the particles can further be achieved or initiated by an alternating magnetic field. Thereby it can be achieved that the thermotherapeutic treatment is yet supported by an anti-proliferative drug immediately in the area of surgery, which will increase effectiveness once again. Of course additional chemotherapy or radiotherapy is possible herein as well concomitantly or time-displaced.

The at least one pharmacological drug does not have to be bound mandatorily to the particles, preferably nanoparticles. It can be additionally contained in the inventive medical product or be applied to its surface without binding to the particles.

Binding of the drug to the particles has the advantage that a rather target-oriented release will occur, as the drug together with the particle can penetrate the cancer cells or can attach to the cancer cells and can be released there induced by a magnetic field.

In this context, “effected” or “induced by a magnetic field” means that for one thing the alternating magnetic field or impulses cause directly the release or detachment, or that the detachment of the drug occurs indirectly for example by activation of enzymes or the generation of heat.

Thus, the nanoparticle-containing medical products in the form of medical cellulose, bandaging materials, wound inserts, surgical suture material, compresses, medical sponges, medical textiles, ointments, gels, or film-forming sprays can yet contain at least one pharmacological drug, preferably an anti-cancer substance. Suitable drugs as well as their binding to the particles are described below in detail.

Said implants and implantable medical products are heated in an alternating magnetic field by applying an external alternating magnetic field after application of the medical products or the biodegradable medical products to the area of surgery.

Heating of the particles occurs in an alternating magnetic field wherein the strength of the alternating magnetic field preferably lies between 1 and 25 kA/m, more preferably between 2 and 18 kA/m, and the frequency lies preferably between 5 to 5,000 kHz, more preferably between 10 and 1000 kHz.

Magnetic particles, preferably superparamagnetic nanoparticles, as well as optionally present drugs are released supported by heating, which then will attach to the cancer cells and kill them. Said sparing therapy form of thermotherapy is applicable particularly in combination with other treatment procedures such as radiotherapy and/or chemotherapy.

### Magnetic Particles

In an embodiment, any magnetic particles can be utilized as long as they can be heated by an alternating magnetic field.

Thus, microparticles and nanoparticles in particular and superparamagnetic micro- and nanoparticles in particular are preferred.

Said nanoparticles feature preferably a magnetic, more preferably a superparamagnetic core. Preferred are materials such as maghemite, magnetite, iron-nickel alloys, nickel-copper alloys or cobalt-nickel alloys such as FeNi or CoNi.

To improve the magnetic characteristics a second magnetic core layer can be applied as well. This will result in a higher total coercive field in comparison to nanoparticles with a one-layered core. The first core layer can be made out of superparamagnetic material, and the second core layer can be made out of a material differing from the one of the first core layer. Further layers which for example will carry drugs can be applied to this core. Multi-layered particles for infiltration of tumor cells by particle-drug-conjugates are described in application WO 98/58673 A.

The core or cores themselves are composed of a magnetic material, preferably a ferromagnetic, anti-ferromagnetic, ferrimagnetic, anti-ferrimagnetic or superparamagnetic material, further preferred made of iron oxide, in particular superparamagnetic iron oxide or of pure iron which is provided with an oxide layer. Such nanoparticles can be heated by an alternating magnetic field with a preferred magnetic field strength between 2 and 25 kA/m and a frequency which lies preferably between 5 and 5000 kHz. Heating of the tissue containing the nanoparticles to more than 50° C. is possible by this method. Such high temperatures can be achieved since iron in the form of nanoparticles can be absorbed up to 800 pg and more per tumor cell. Therefore the nanoparticles cannot leave the target area over a longer period of time and heat can be—also repeatedly—applied in the tumor in this way very precisely and without contact from the outside. Heating is based on the release of translation and rotation heat as a result of magnetic relaxation processes as well as losses of hysteresis heat.

The nanoparticles are preferably composed of iron oxide and in particular of magnetite (Fe₃O₄), maghemite (γ-Fe₂O₃) or mixtures of both oxides. In general, the preferred nanoparticles can be defined by the formula FeO_{X}, wherein x means a rational number of 1 to 2. The nanoparticles feature a diameter of preferably less than 500 nm. The nanoparticles preferably have a mean diameter of 15 nm or lie preferably within the range of 1-200 nm and in particular preferably in the range of 5-30 nm.

Production of nanoparticles without drug and also without coating is described in detail in DE 4428851 A.

Besides magnetic materials of the formula FeO_{X} wherein x is a rational number in the range of 1.0 to 2.0, materials of the general formula MFe₂O₄ with M=Co, Ni, Mn, Zn, Cd, Ba or other ferrites can be used.

It is also possible to configure nanoparticles with another metallic core instead of iron oxide. Herein the metals gold, silver, platinum, copper, cobalt, nickel, iron, manganese, samarium, neodymium, iridium, osmium, ruthenium, rhodium, palladium or alloys of the above listed metals are to be named.

But it is also possible to produce the nanoparticles from a non-magnetic material such as silicon dioxide (SiO₂). Further, silica or polymer particles, in which magnetic materials such as the magnetic materials mentioned above are incorporated and/or attached to, are likewise suitable.

Furthermore, the magnetic particles can be derivatized to that effect that chemical structures such as antibodies, nucleic acids, peptides, aptamers or other molecules with target-finding properties are present on the surface of the particles which will enhance the affinity of the particles to degenerated cells. Such surface modifications improve the affinity to cancer cells due to recognition of specific surface structures on the degenerated cells. Preferred chemical structures which will confer target-finding properties to the magnetic particles are for example polyclonal antibodies, monoclonal antibodies, humanized antibodies, human antibodies, chimeric antibodies, recombinant antibodies, bi-specific antibodies, antibody fragments, aptamers, Fab fragments, Fc fragments, peptides, peptidomimetics, gapmers, ribozymes, CpG oligomers, DNAzyme, riboswitches as well as lipids.

In an embodiment the nanoparticles can optionally be bound to therapeutic active substances. Bonding of the drug can take place covalently or by prevailingly covalent bonding and/or sufficiently strong ionic bonding, inclusion compound or complex bonding so that an uncontrolled release of the drug is prevented to a great extent. Detachment of the drug without impact of an alternating magnetic field is understood as uncontrolled release.

Anti-proliferative, anti-migratory, anti-angiogenic, anti-thrombotic, anti-inflammatory, anti-phlogistic, cytostatic, cytotoxic, anti-coagulative, anti-bacterial, anti-viral and/or anti-mycotic drugs can be selected as therapeutically active substances wherein anti-proliferative, anti-migratory, anti-angiogenic, cytostatic and/or cytotoxic drugs as well as nucleic acids, amino acids, peptides, proteins, carbohydrates, lipids, glycoproteins, glycans or lipoproteins with anti-proliferative, anti-migratory, anti-angiogenic, anti-thrombotic, anti-inflammatory, anti-phlogistic, cytostatic, cytotoxic, anti-coagulative, anti-bacterial, anti-viral and/or anti-mycotic properties are preferred. Furthermore, these substances can be radiosensitizers or sensitizers or enhancers of other—also combined—conventional cancer treatment methods or contain such sensitizers.

Alkylating agents, antibiotics with cytostatic properties, anti-metabolites, microtubule inhibitors and topoisomerase inhibitors, platinum-containing compounds and other cytostatics such as asparaginase, tretinoin, alkaloids, podophyllum toxins, taxanes and Miltefosin®, hormones, immune modulators, monoclonal antibodies, signal transducers (signal transduction molecules) and cytokines among others can be used as cytotoxic and/or cytostatic compounds, i.e. chemical compounds with cytotoxic and/or cytostatic properties.

As examples for alkylation agents can be named a.o. chlorethamine, cyclophosphamide, trofosfamide, ifosfamide, melphalan, chlorambucil, busulfan, thiotepa, carmustin, lomustin, dacarbazine, procarbazine, temozolomide, treosulfan, estramustin and nimustin.

Examples for antibiotics with cytostatic properties are daunorubicin, doxorubicin (adriamycin), dactinomycin, mitomycin C, bleomycin, epirubicin (4-epi-adriamycin), idarubicin, mitoxantrone and amsacrin.

Methotrexate, 5-fluorouracil, 6-thioguanine, 6-mercaptopurine, fludarabine, cladribine, pentostatin, gemcitabine, azathioprin, raltitrexed, capecitabine, cytosinarabinoside, thioguanine and mercaptopurin can be named as examples for anti-metabolites (anti-metabolic drugs).

Vincristin, vinblastin, vindesin, etoposid as well as teniposid among others belong to the class of alkaloids and podophyllum toxins. Furthermore, platinum-containing compounds can be used. Cisplatin, carboplatin and oxaplatin are mentioned as platinum-containing compounds, for example. For example, alkaloids such as vinca alkaloids (vincristin, vinblastin, vindesin, venorelbin) and taxanes (paclitaxel/Taxol®, paclitaxel and docetaxel) as well as derivatives of paclitaxel belong to microtubule inhibitors. Podophyllum toxins (etoposide, teniposide) and camptotheca alkaloids (camptothecin, topotecan and irinotecan) can be quoted as topoisomerase inhibitors.

For example, hydrocarbamides (hydroxyurea), imatinib, Miltefosin®, amsacrin, pentostatin, bexaroten, tretinoin and asparaginase count as other cytostatic drugs (other cytostatics). Representatives of the compound class of monoclonal antibodies are trastuzumab (also known as Herceptin®), alemtuzumab (also known as MabCampath®) and rituximab (also known as MabThera®).

In some embodiments hormones such as glucocorticoids (prednisone), estrogens (fosfestrol, estramustin), LHRH (buserelin, goserelin, leuprorelin, triptorelin), flutamide, cyproteronacetate, tamoxifen, toremifen, aminoglutethimide, formestan, exemestan, letrozol and anastrozol can be used. Interleukin-2, interferon-α, interferon-β, erythropoietin, G-CSF, trastuzumab (Herceptin®), rituximab (MabThera®), efitinib (Iressa®), ibritumomab (Zevalin®), levamisol as well as retinoids belong to the classes of immunomodulators, cytokines, antibodies and signal transducers.

The drugs mentioned above can be contained together with the magnetic particles in the inventive medical product or are applied to its surface. In the case that the drug is bound covalently or ionically to the magnetic particles or the medical product or the biodegradable medical product, binding of the drug occurs by e.g. hydroxy groups, amino groups, carbonyl groups, thiol groups, or carboxy groups, depending on which functional groups the respective drug is carrying.

Hydroxy groups are bound preferably as ester, acetal or ketal, thiol groups preferably as thiol ester, thiol acetal or thiol ketal, amino groups preferably as amides and partly as imines (Schiff bases), carboxy groups preferably as esters or amides and carbonyl groups preferably as ketals.

Moreover, it is preferred to bind the drug or drugs not directly to a nanoparticle or the medical product or the biodegradable medical product but to immobilize it by a linker molecule. Further, fictionalization of the surface of the nanoparticle is known so that amino groups, hydroxy groups, carboxy groups or carbonyl groups can be generated on the surface of the nanoparticles by known methods.

The therapeutically active substances are bound to the nanoparticles and/or the medical product or the biodegradable medical product directly or by a linker molecule, preferably via amide bonding or ester bonding.

Linkers are preferred which contain pH-cleavable acetal, ester, hydrazone or imine groups and can be cleaved by an acidic or enzymatic reaction.

The amide group is to be named as an enzymatically cleavable group in or at the linker molecule. Groups cleavable thermally or via acid include e.g. phosphate groups, thio phosphate groups, sulphate groups, phosphamide groups, carbamate groups or imine groups.

The drug does not necessarily need to be bound covalently to the linker or the biodegradable medical product but can be bound ionically or via hydrogen bonds or can be present in intercalated or coordinated form.

As described before, any magnetic particles can be utilized for the inventive medical products. Examples for such magnetic particles are described in WO 2005 070471 A2, WO 02/43708 A2, U.S. Pat. No. 5,411,730 A1, WO 2005 042142 A2, WO 03/026618 A1, WO 2005 065282 A2, WO 2006 108405 A2 and WO 2007 019845 A2.

### Biodegradable Medical Products

The inventive biodegradable medical products in the form of implants, gels, tissues, textile, wound coating or film-forming preparations remain inside the body of the patient after closure of the wound after cancer surgery by a surgeon.

The inventive biodegradable medical products serve in particular for the after-treatment of the operative field by heat generated through thermotherapy for killing remaining tumor cells and for the prevention of recurrences.

Thus, the inventive biodegradable medical products are composed of physiologically acceptable materials and/or are cleaved into physiologically acceptable degradation products and components.

Materials for the inventive medical products are selected from the group including or consisting of: Polyacrylic acid, polyacrylate, polymethyl methacrylate, polybutyl methacrylate, polyisobutyl methacrylate, polyacrylamide, polyacrylnitrile, polyamide, polyetheramide, polyethyleneamine, polyimide, polycarbonate, polycarbourethane, polyvinylketone, polyvinylhalogenide, polyvinylidenhalogenide, polyvinylether, polyvinyl aromatics, polyvinyl ester, polyvinylpyrollidone, polyoxymethylene, polyethylene, polypropylene, polytetra-fluoroethylene, polyurethane, polyolefin elastomer, polyisobutylene, EPDM gums, fluorosilicone, carboxymethylchitosan, polyethyleneterephtalate, polyvalerate, carboxymethylcellulose, cellulose, rayon, rayon triacetate, cellulose nitrate, cellulose acetate, hydroxyethyl cellulose, cellulose butyrate, cellulose acetate-butyrate, ethylvinylacetate copolymer, polysulfone, polyethersulfone, epoxy resin, ABS resins, EPDM gums, silicone pre-polymer, silicone, polysiloxane, polyvinyl halogen, cellulose ether, cellulose triacetate, chitosane, chitosan derivatives, polymerisable oils, polyvalerolactones, poly-ε-decalacton, polylactide, polyglycolide, co-polymers of polylactide and polyglycolide, poly-ε-caprolactone, polyhydroxy butyric acid, polyhydroxybutyrate, polyhydroxyvalerate, polyhydroxybutyrate-co-valerate, poly(1,4-dioxan-2,3-dione), poly(1,3-dioxan-2-one), poly-para-dioxanone, polyanhydride, polymaleic acid anhydride, polyhydroxy methacrylate, polycyanoacrylate, polycaprolacton dimethylacrylate, poly-β-maleic acid, polycaprolactonbutyl acrylate, multi-block polymers made of oligocaprolactonediol and oligodioxanondiol, polyetherester-multi-block polymers made of PEG and poly(butyleneterephthalate), polypivotolactone, polyglycolic acid trimethylcarbonate, polycaprolactone-glycolide, poly(γ-ethylglutamate), poly(DTH-iminocarbonate), poly(DTE-co-DT-carbonate), poly(bisphenol A-iminocarbonate), polyorthoester, polyglycolic acid trimethyl-carbonate, polytrimethylcarbonate, polyiminocarbonate, polyvinylic alcohols, polyester amides, glycolidized polyesters, polyphosphoesters, polyphosphazenes, poly[p-carboxyphenoxy)propane], polyhydroxypentaic acid, polyethylene oxide-propylene oxide, soft polyurethanes, polyurethanes with amino acid rests in the backbone, polyether esters, polyethylene oxide, polyalkenoxalates, polyorthoesters, carrageenans, starch, collagen, protein-based polymers, polyamino acids, synthetic polyamino acids, zein, modified zein, polyhydroxyalkanoates, pectic acid, actinic acid, fibrin, modified fibrin, casein, modified casein, carboxymethylsulphate, albumin, hyaluronic acid, heparan sulphate, heparin, chondroitin sulphate, dextrane, cyclodextrine, co-polymers made of PEG and polypropyleneglycol, gum arabic, guar, or other gum resins, gelatine, collagen, collagen-N-hydroxysuccinimide, lipids, lipoids, polymerisable oils and their modifications, co-polymers and mixtures of the aforementioned substances.

The aforementioned polymers are biodegradable and can be produced in polymerization grades and cross-linkages which are biodegradable.

It is understood by the term “biodegradable” or “bioresorbable” that these materials are degraded or will have been degraded up to 90 percent by weight within a period of 1 month up to 12 months, preferably up to 6 months, under physiological conditions.

Preferred biodegradable polymers are polylactides, polyglycolides, co-polymers of polylactides and polyglycolides, polyhydroxybutyrate, polyhydroxymethacrylate, polyorthoesters, glycolidised polyesters, polyvinylic alcohols, polyvinylpyrrolidone, acrylamide acrylic acid co-polymers, hyaluronic acid, heparan sulphate, heparin, chondroitin sulphates, dextran, β-cyclodextrins, hydrophilically cross-linked dextrins, alginates, phospholipides, carbomers, cross-linked peptides and proteins, silicones, polyethylene glycol (PEG), polypropylene glycol (PPG), co-polymers of PEG and PPG, collagen, polymerizable oils and waxes, and mixtures and co-polymers thereof.

Furthermore, polyesters, polylactides as well as co-polymers of diols and esters or diols and lactides are preferred. For example, ethane-1,2-diol, propane-1,3-diol or butane-1,4-diol are used as diols.

In an embodiment polyesters are utilized for the polymeric layer. Such polymers of the group of polyesters are preferred which will feature the following repetitive units:

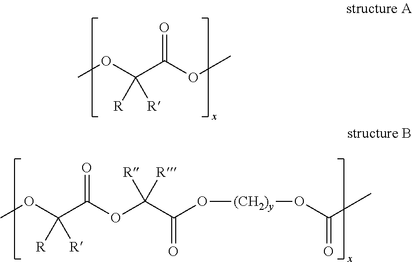

In the depicted repetitive units, R, R′, R″ and R′″ define alkyl groups with 1 to 5 carbon atoms, in particular methyl, ethyl, propyl, isopropyl, n-butyl, s-butyl, t-butyl, iso-butyl, n-pentyl or cyclopentyl and preferably methyl or ethyl. Y is an integer of 1 to 9, and x means the polymerisation grade. In particular, the following polymers with the shown repetitive units are preferred:

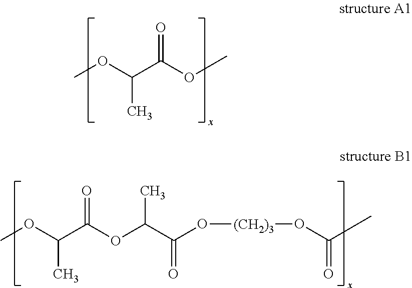

As further representatives of resorbable polymers shall be named Resomer®, poly(L-lactide)s of the general formula —(C₆H₈O₄)ₙ— such as L 210, L 210 S, L 207 S, L 209 S, poly(L-lactic-co-D,L-lactide)s of the general formula —(C₆H₈O₄)ₙ— such as LR 706, LR 708, L 214 S, LR 704, poly(L-lactic-co-trimethylcarbonate)s of the general formula —[(C₆H₈O₄)ₓ—(C₄H₆O₃)_{y}]ₙ— such as LT 706, poly(L-lactic-co-glycolide)s of the general formula —[(C₆H₈O₄)ₓ—(C₄H₄O₄)_{y}]ₙ— such as LG 824, LG 857, poly(L-lactic-co-ε-caprolactone)s of the general formula —[(C₆H₈O₄)ₓ—(C₆H₁₀O₂)_{y}]ₙ— such as LC 703, poly(D,L-lactic-co-glycolide)s of the general formula —[(C₆H₈O₄)ₓ—(C₄H₄O₄)_{y}]ₙ— such as RG 509 S, RG 502 H, RG 503 H, RG 504 H, RG 502, RG 503, RG 504, poly(D,L-lactide)s of the general formula —[(C₆H₈O₄)ₙ— such as R 202 S, R 202 H, R 203 S and R 203 H. Resomer® 203 S is herein the successor of the particularly preferred polymer, Resomer® R 203. The name Resomer® stands for a high-tech product of the company Boehringer Ingelheim.

Basically, the use of absorbable polymers is particularly preferred. Further, homo-polymers of lactic acid (polylactides) as well as polymers which are produced out of lactic and glycolic acids are preferred.

### Bio-Stable Medical Products

Inventive bio-stable or non-biodegradable medical products in the forms of gels, sponges and in particular film-forming preparations, film-forming sprays or textiles, tissues, cellulose, wound covers and the like are produced from non-biodegradable or poorly biodegradable material.

Materials for the inventive bio-stable medical products are selected from the group including or consisting of: Polyacrylic acid and polyacrylate such as polymethylmethacrylate, polybutylmethacrylate, polyacrylamide, polyacrylonitriles, polyamides, polyetheramides, polyethyleneamine, polyimides, polycarbonates, polycarbourethanes, polyvinylketones, poly(vinyl halogenide)s, poly(vinylidene halogenide)s, polyvinylethers, polyvinylic aromatics, polyvinylic esters, polyvinylpyrollidones, polyoxymethylenes, polyethylene, polypropylene, polytetra-fluoroethylene, polyurethanes, polyolefin elastomers, polyisobutylene, EPDM gums, fluorosilicones, carboxymethyl chitosan, polyethyleneterephtalate, polyvalerate, carboxymethyl cellulose, cellulose, rayon, rayon triacetates, cellulose nitrate, cellulose acetate, hydroxyethyl cellulose, cellulose butyrate, cellulose acetate-butyrate, ethylvinylic acetate-co-polymeres, polysulfones, epoxy resins, ABS resins, EPDM gums, silicones such as polysiloxanes, polyvinylic halogens and co-polymers, cellulose ether, cellulose triacetate, chitosan and co-polymers and/or mixtures thereof.

Preferred bio-stable polymers, which are used in medical engineering and for bio-stable implants, are polyethersulfone, substituted polyethersulfone, polyphenylsulfone, substituted polyphenylsulfone, polysulfone block co-polymers, perfluorinated polysulfone block co-polymers, semi-fluorinated polysulfone block co-polymers, substituted polysulfone block co-polymers and/or mixtures of the aforementioned polymers.

### Gels

The inventive nanoparticles can be incorporated into gels or hydrogels, too, or be components of film-forming spays which preferably are biodegradable as well. For better stabilization of the gels or film-forming sprays the inventive nanoparticles described herein can be combined with gelling or film-forming agents.

Suitable gelling or film-forming agents preferably are cellulose-based materials such as cellulose nitrate or ethyl cellulose or physiologically safe polymers thereof, polyvinylacetate, partially saponified polyvinylacetate, polymer mixtures of vinylic acetate and acrylic acid or crotonic acid or maleic acid monoalkyl ester, ternary polymer mixtures of vinylic acetate and crotonic acid and vinyl neodecanoate, or crotonic acid and vinylic propionate, polymer mixtures of methylvinylic ether and maleic acid monoalkyl ester, in particular as maleic acid monobutyl ester, polymer mixtures of fatty acid vinylic ester and acrylic acid or methacrylic acid, polymer mixtures of N-vinylpyrrolidone, methacrylic acid and methacrylic acid alkyl ester, polymer mixtures of acrylic acid and methacrylic acid or acrylic acid alkyl ester or methacrylic acid alkyl ester, in particular with a content of quarternary ammonium groups, or polymers, co-polymers or mixtures containing ethyl acrylate, methyl methacrylate or trimethylammonioethyl methacrylate chloride, or polyvinylic acetals and polyvinylic butyrals, alkyl-substituted poly-N-vinylpyrrolidones, alkyl ester of polymer mixtures of olefins and maleic acid anhydride, reaction products of colophonium with acrylic acid and styrax resins, chitosan, Luvimer 100®, aluminium stearate, carbomers, cocamide MEA, carboxymethyldextrane, carboxymethyl hydroxypropyl guar or red algae carrageenans.

Alkyl radicals of the aforementioned esters are usually short-chain and mostly don't have more than four carbon atoms. Such compounds are designated herein as polymer-forming or gelling agents.

Moreover, water-soluble polymers such as ionic polyamides, polyurethanes and polyesters as well as homo- and co-polymers of ethylenic unsaturated monomers belong to the gelling and film-forming agents, respectively. Such compounds are for example the brands Acronal®, Acudyne°, Amerhold®, Amphome®, Eastman AQ®, Ladival®, Lovocryl®, Luviflex VBM®, Luvimer®, Luviset P. U. R.®, Luviskol®, Luviskol Plus®, Stepanhold®, Ultrahold®, Ultrahold Strong® or Versatyl®. Luvimer® is a polyacrylate.

Further components of the inventive gels can be above all natural polymers. Among them are albumin, collagen, hyaluronan, chitosan and chitin. A co-polymer or block co-polymer of polyethylene oxide with terminal α-hydroxy acids or poly-α-hydroxy acids is a particularly preferred non-natural polymer.

Furthermore, glycosaminoglycans such as aggrecan, decorin, biglycan and fibromodulin are common components of bioabsorbable gels or film-forming solutions or sprays.

Salt solutions such as saline solution (0.9 percent), PBS (phosphate buffered saline, i.e. phosphate buffered saline solution), DMEM (Dulbecco's Modified Eagle Medium) can be used in the gels, solutions and sprays, too.

For the use of superparamagnetic particles with an iron oxide core a content of iron oxide of 3-30 percent by weight in 200 mg gel is preferred, a content of iron oxide of 5-25 percent by weight in 200 mg gel is more preferred and a content of iron oxide of 10-20 percent by weight in 200 mg gel is particularly preferred.

### Polymeric Carriers

The magnetic particles can be added already during the production of the polymers and will then be incorporated into the bioresorbable polymeric structure.

Examples for biodegradable medical products are polymeric beads containing the magnetic particles. The polymer beads preferably are composed of polyhydroxybutyrate, polylactide, polyglycolide or co-polymers of polylactide-co-glycolide. Alginate as well as Eudragit® are another particularly preferred material. These polymer beads contain magnetic particles up to 20 percent by weight.

The polymer beads can be used as such or can be incorporated into gels or pastes or can be immobilized to medical cellulose.

The polymer beads can be heated up to a temperature of 50° C. in an alternating magnetic field.

### Medical Cellulose

The coated medical implantable products onto which the nanoparticles are applied preferably are bioresorbable. That is, they can be completely dissolved in the body or at least are physiologically well-tolerated.

The medical implants containing the nanoparticles are medical cellulose, bandaging materials, wound inserts, surgical sutures, compresses and medical textiles, among others.

Polyhydroxybutyrate and cellulose derivatives, chitosan derivatives as well as collagen, polyethylene glycol, polyethylene oxide and polylactides are preferred materials for medical cellulose and textiles. Calcium alginate products interwoven with sodium carboxymethyl cellulose are used preferably if alginates are used as wound covers. SeaSorb Soft from the company Coloplast is to be given as an example.

If the nanoparticles are applied to bandages and/or wound inserts the products Tabotamp® and Spongostan® from the company Johnson and Johnson have to be mentioned in particular. These products are produced of regenerated cellulose by controlled oxidation.

If surgical sutures are to be impregnated with the nanoparticles surgical sutures are used that are composed of polyglycolic acid, polycaprolactone-co-glycolide or poly-p-dioxanone. Examples are the products Marlin®, PCL and Marisorb® from the company Catgut GmbH.

If compresses are to be impregnated with the nanoparticles in particular sterile gauze compresses of 100% cotton have to be used herein. Examples are the product lines Stericomp® and Askina®.

If medical cellulose is used it is preferred that it has a cellulose content of more than 90%.

Trevira® products are preferred if medical textiles are used.

The medical textiles and cellulose are sprayed with a solution of the magnetic particles in water, ethanol or mixtures of water and ethanol or are dipped therein. The dipping or spraying process can be repeated several times after drying of the medical product.

10 μg to 100 mg of magnetic particles are applied per cm² surface of the medical product.

For each gram of the medical product 100 μg to 2 g of magnetic particles are coated.

### Sponges

The medical sponges are bioresorbable implants with a spongy porous structure.

Preferred materials for medical sponges are collagen, oxidized cellulose, chitosan, thrombin, fibrin, chitin, alginate, hyaluronic acid, PLGA, PGA, PLA, polysaccharides and globin.

If medical sponges are used it is preferred that they have a collagen content of more than 90%.

For each gram of the medical product 100 μg to 2 g of magnetic particles are applied.

### Ointments and Pastes

If the nanoparticles are incorporated into ointments a basis of the ointment will be used the includes purified water in an amount of preferably 5-50 percent by weight, more preferred of 10-40 percent by weight and most preferred of 20-30 percent by weight. In addition, the ointment yet contains petroleum jelly in an amount of preferably 40-90 percent by weight, more preferred of 50-80 percent by weight and most preferred of 20-60 percent by weight. In addition, the ointment may contain viscous paraffin in an amount of preferably 5-50 percent by weight, more preferred of 10-40 percent by weight and most preferred of 20-30 percent by weight.

Moreover, gelling and/or film-forming agents as described herein can be added in an amount of up to 30 percent by weight. In addition, polymers such as cellulose, chitosan, thrombin, fibrinogen, chitin, alginates, albumin, hyaluronic acid, hyaluronan, polysaccharides, globin, polylactide, polyglycolide, polylactide-co-glycolide, polyhydroxybutyrates, cellulose derivatives, chitosan derivatives, polyethylene glycol and polyethylene oxide in amounts of up to 30 percent by weight can be used.

### Film-Forming Sprays

The nanoparticles may be incorporated into spraying solutions or can be components of film-forming sprays. The magnetic particles or drug-containing nanoparticles described herein can be used in combination with gelling or film-forming agents for a better stabilization of the film-forming sprays. Film-forming sprays contain at lease one or more film agents.

Suitable film-forming agents preferably are compounds on a cellulose basis such as cellulose nitrate or ethyl cellulose or physiologically safe polymers thereof, polyvinyl acetate, partially saponified polyvinyl acetate, polymer mixtures of vinyl acetate and acrylic acid or crotonic acid or maleic acid monoalkyl ester, ternary polymer mixtures of vinyl acetate and crotonic acid and vinyl neodecanoate, or crotonic acid and vinylic propionate, polymer mixtures of methylvinylic ether and maleic acid monoalkyl ester, in particular as maleic acid monobutyl ester, polymer mixtures of fatty acid vinylic ester and acrylic acid or methacrylic acid, polymer mixtures of N-vinylpyrrolidone, methacrylic acid and methacrylic acid alkyl ester, polymer mixtures of acrylic acid and methacrylic acid or acrylic acid alkyl ester or methacrylic acid alkyl ester, in particular with a content of quaternary ammonium groups, or polymers, co-polymers or mixtures containing ethyl acrylate, methyl methacrylate or trimethylammonioethyl methacrylate chloride, or polyvinylic acetals and polyvinylic butyrals, alkyl-substituted poly-N-vinylpyrrolidones, alkyl ester of polymer mixtures of olefins and maleic acid anhydride, reaction products of colophonium with acrylic acid and styrax resins, chitosan, Luvimer 100®, aluminium stearate, carbomers, cocamide MEA, carboxymethyldextrane, carboxymethyl hydroxypropyl guar or red algae carrageenans.

Alkyl radicals of the aforementioned esters are usually short-chain and mostly don't have more than four carbon atoms.

Moreover, water-soluble polymers such as ionic polyamides, polyurethanes and polyesters as well as homo- and co-polymers of ethylenic unsaturated monomers belong to the gelling and film-forming agents, respectively. Such compounds are for example the brands Acronal®, Acudyne®, Amerhold®, Amphome®, Eastman AQ®, Ladival®, Lovocryl®, Luviflex VBM®, Luvimer®, Luviset P. U. R.®, Luviskol®, Luviskol Plus®, Stepanhold®, Ultrahold®, Ultrahold Strong® or Versatyl®. Luvimer® is a polyacrylate that was developed as a hair styling polymer by BASF AG.

Preferred solvents are water, ethanol or mixtures of water and ethanol.

For the use of superparamagnetic particles with an iron oxide core a content of iron oxide of 3-30 percent by weight in 200 mg gel is preferred, a content of iron oxide of 5-25 percent by weight in 200 mg gel is more preferred and a content of iron oxide of 10-20 percent by weight in 200 mg gel is most preferred.

For each gram of the medical product 100 μg to 2 g of magnetic particles are applied.

Manufacture of the nanoparticle-containing implants occurs by dipping or spraying processes. Herein the products to be implanted are dipped in a nanoparticle-containing solution or suspension or are sprayed with a nanoparticle-containing solution. After that the products are dried and aseptically packed. Gels, ointments, solutions and sprays are obtained by producing the desired pharmaceutical preparation according to standard procedures and the desired amount of magnetic particles is preferably added in a last step.

The obtained inventive biodegradable medical products are used for treatment and prophylaxis of tumors, carcinoma and cancer and serve in particular for the after-treatment of a surgical area after cancer surgery and in particular after removal of a solid tumor.

Examples of cancer and tumors for which the inventive medical products can be used are: Adenocarcinomas, choroid melanoma, acute leukemia, acoustic neurinoma, ampullary carcinoma, anal carcinoma, astrocytomas, basalioma, pancreatic carcinoma, connective tissue tumor, bladder cancer, bronchial carcinoma, non-small cell bronchial carcinoma, breast cancer, Burkitt's lymphoma, corpus carcinoma, CUP syndrome, colon cancer, cancer of the small intestine, small intestine tumors, ovarian cancer, endometrial carcinoma, ependymoma, epithelial cancers, Ewing's sarcoma, gastrointestinal tumors, gall-bladder cancer, bilary carcinomas, uterine cancer, cervical cancer, glioblastomas, gynecological tumors, otorhinolaryngologic tumors, hematologic neoplasias, urethral cancer, skin cancer, brain tumors (gliomas), brain metastases, testicular cancer, hypophyseal tumor, carcinoids, Kaposi's sarcoma, laryngeal cancer, germinal tumor, bone cancer, colorectal carcinoma, head-neck tumors (tumors of neck, nose and ear areas), colon carcinoma, craniopharyngiomas, cancer of the mouth area and the lips, hepatic cancer, hepatic metastases, eyelid tumor, lung cancer, lymphatic gland cancer (Hodgkin/Non-Hodgkin), lymphomas, stomach cancer, malignant melanoma, malignant neoplasia, malignomas of the gastrointestinal tract, mammary carcinoma, rectal cancer, medulloblastomas, melanoma, meningeomas, Hodgkin's disease, mycosis fungoides, nose cancer, neurinoma, neuroblastoma, kidney cancer, renal cell carcinoma, Non-Hodgkin's lymphomas, oligodendroglioma, esophageal carcinoma, osteolytic carcinoma and osteoplastic carcinoma, osteosarcoma, ovarian carcinoma, pancreatic carcinoma, penile cancer, squamous cell carcinomas of the head and neck, prostate cancer, pharyngeal cancer, rectal carcinoma, retinoblastoma, vaginal cancer, thyroid carcinoma, Schneeberger's disease, esophageal cancer, spinalioma, T-cell lymphoma (mycosis fungoides), thymoma, tubal carcinoma, eye tumors, urethral cancer, urologic tumors, urothelial carcinoma, vulvar cancer, mastoid involvement, soft tissue tumors, soft tissue sarcoma, Wilms' tumor, cervix carcinoma and tongue cancer.

In particular, solid tumors are preferred. Further preferred are prostate carcinoma, brain tumors, sarcomas, cervical carcinomas, ovarian carcinomas, mammary carcinomas, bronchial carcinomas, melanomas, head-neck tumors, esophageal carcinomas, rectal carcinomas, pancreatic carcinomas, bladder carcinomas, renal carcinomas, metastases in the liver, brain and lymphatic nodes.

Further, use and application of the inventive bioresorbable medical products are particularly preferred in the field of medicine preferably in conjunction with radiotherapy and/or together with conventional chemotherapy.

This sparing method of thermotherapy includes a locally limited application of cancer drugs and thus reduces drug burden and adverse effects for the patient. Moreover, the probability of recurring metastasis will be strongly decreased as cancer combat of tumor cells remaining after incomplete resection occurs locally and selectively. Moreover, the drugs optionally located on the inventive implant or medical product can be detached from the nanoparticle by an alternating magnetic field applied from the outside and will have a more selective effect straight at the site of activity. This will allow for a more precise drug dosage as no drugs will be lost during the transport through the body due to the localized therapy method. The method described above can be also effectively carried out against cancer cells with nanoparticles without attached drug. Herein nanoparticles attach to the cancer cells or penetrate the cancer cells and destroy the cancer cells by a magnetic field applied from the outside heating the magnetic particles.

Additionally, molecules with target-finding properties such as monoclonal antibodies and/or aptamers may be coupled to the surface of the nanoparticles or the outer layer or shell of the nanoparticles for a further increase in affinity to specific cell types.

In an embodiment the cores of the magnetic nanoparticles are composed of magnetite (Fe₃O₄), maghemite (γ-Fe₂O₃) or mixtures of both oxides and preferably are superparamagnetic. Furthermore, the cores are stabilized by colloidal protective shells which will allow attachment of the therapeutically effective agents.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1A

### General Instructions for the Production of a Nanoparticle Suspension/Solution for Impregnation or Spraying or Dipping of the Carrier

A solution of 0.23 Mol FeCl₂ and 0.46 Mol FeCl₃ in 1 l of water is degassed by nitrogen. Thereupon as much of 5 M NaOH is added within 20 minutes that a pH-value of 11.5 is reached. The resulting precipitate is heated to 65° C. for ten minutes and subsequently will be cooled to room temperature within five minutes. After that, the precipitate is suspended in deionized and degassed water until a pH-value of the washing solution of 9 will be reached. The precipitate is suspended in water and the suspension is adjusted to a pH-value of 6 with glacial acetic acid. 10 percent by volume of a 30 percent by weight aqueous H₂O solution are added to the resulting suspension which after that will be stirred until termination of gas development. Thereupon the suspension will be diluted with water to a content of solid iron oxide of 5 percent by weight.

### Example 1B

### Without Oxidation/with Air Gassing

0.1 mol FeCl₃×6H₂O and 0.2 mol FeCl₃ (water-free), 50 g sodium acetate and 195 g diaminohexane in 900 ml ethylene glycol were dissolved for the production of iron oxide nanoparticles in ethylene glycol and were heated to 60° C. for 1 hour. Then, the solution was heated to boiling point within 30 minutes. The boiling temperature was maintained for six hours. The resulting dispersion was cooled slowly to room temperature.

The particles were washed three times with a mixture of ethanol and water.

After that, the particles were resuspended in 900 ml ethylene glycol and were gassed with atmospheric oxygen. The suspension was heated to the boiling point of ethylene glycol and was kept at this temperature for 24 hours.

After cooling the particles were washed with water/ethanol and suspended in water.

These particles were coated in an analogous manner to example 1G.

### Example 1C

### With Oxidation/with Air Gassing

0.1 mol FeCl₃×6H₂O and 0.2 mol FeCl₃ (water-free), 50 g sodium acetate and 195 g diaminohexane in 900 ml ethylene glycol were dissolved for the production of iron oxide nanoparticles in ethylene glycol and were heated to 60° C. for one hour. Then, the solution was heated to boiling point within 30 minutes. Boiling temperature was maintained for six hours. The resulting dispersion was cooled slowly to room temperature.

The particles were washed three times with a mixture of ethanol and water.

After that, the particles were resuspended in 900 ml ethylene glycol and gassed with atmospheric oxygen. The suspension was heated to the boiling point of ethylene glycol and was kept at this temperature for 24 hours.

After cooling the particles were washed with water/ethanol and suspended in 900 ml 1 M HNO₃. Then, 450 ml of 0.7 M ferrous nitrate solution (Fe(NO₃)₃×9 H₂O) were added and boiled under reflux for one hour (100° C.). The particles were washed three times with 500 ml water each. These particles were coated in an analogous manner to example 1G.

### Example 1D

### Without Oxidation/without Air Gassing

0.1 mol FeCl₃×6H₂O and 0.2 mol FeCl₃ (water-free), 50 g sodium acetate and 195 g diaminohexane in 900 ml ethylene glycol were dissolved for the production of iron oxide nanoparticles in ethylene glycol and were heated to 60° C. for one hour.

Then, the solution was heated to boiling point within 30 minutes. Boiling temperature was maintained for six hours. The resulting dispersion was cooled slowly to room temperature.

The particles were washed three times with a mixture of ethanol and water.

After that the particles were resuspended in 900 ml ethylene glycol.

The suspension was heated to the boiling point of ethylene glycol and was kept at this temperature for 24 hours.

After cooling the particles were washed with water/ethanol and suspended in water.

These particles were coated in an analogous manner to example 1G.

### Example 1E

### With Oxidation/without Air Gassing

0.1 mol FeCl₃×6H₂O and 0.2 mol FeCl₃ (water-free), 50 g sodium acetate and 195 g diaminohexane in 900 ml ethylene glycol were dissolved for the production of iron oxide nanoparticles in ethylene glycol and were heated to 60° C. for one hour.

Then, the solution was heated to boiling point within 30 minutes. Boiling temperature was maintained for six hours. The resulting dispersion was cooled slowly to room temperature.

The particles were washed three times with a mixture of ethanol and water.

After that, the particles were resuspended in 900 ml ethylene glycol. The suspension was heated to the boiling point of ethylene glycol and kept at this temperature for 24 hours.

After cooling, the particles were washed with water/ethanol and suspended in 900 ml of 1 M HNO₃. Then, 450 ml of 0.7 M ferrous nitrate solution (Fe(NO₃)₃×9 H₂O) were added and boiled under reflux for one hour (100° C.). The particles were washed three times with 500 ml water each.

These particles were coated in an analogous manner to example 1G.

### Example 1F

A solution of 96 g sodium hydroxide and 680 ml oleic acid in 2000 ml methanol was added to a solution of 216 g iron (III) chloride hexahydrate in 500 ml methanol for the production of iron oxide nanoparticles. The resulting solid was washed with methanol and dissolved in diethyl ether. Then it was extracted with water for several times. The solid was precipitated with acetone, washed and vacuum-dried.

75 g of this solid were dissolved in 250 m trioctylamine and heated to 120° C. for one hour.

Then, the solution was heated to 380° C. within 30 minutes in an autoclave. This temperature was kept for 4 hours. The resulting dispersion was cooled slowly to room temperature.

The particles were washed three times with a mixture of ethanol and water.

After that, the particles were suspended in 300 ml diethylene glycol dibutyl ether and were gassed with atmospheric oxygen. The suspension was heated to 300° C. in an autoclave and kept at this temperature for 24 hours.

These particles were oxidized as in example 1C and subsequently coated in an analogous manner to example 1G.

### Example 1G

The particles of examples 1B to 1F were collected by centrifugation at high g forces and were washed with ethanol. 500 mg of the washed product were weighed into an extraction shell (603 g, Whatman) and inserted into a Soxhlet apparatus. 200 ml of ethanol were filled into the still pot of the Soxhlet apparatus as extracting agent. The extracting agent was heated to boiling. Continuous extraction was performed over 8 hours and included ca. 16 extraction cycles. In the course of this the ethanol solution stains yellowish. The extraction shell was removed after termination and the powder transferred to a Schlenk apparatus and vacuum-dried for 1 hour. 0.5 g of the nanoparticle powder of example 4 were suspended in 20 ml of 0.01 M HCl for dispersion of the particles after extraction. Then, the nanoparticles were treated with ultrasound for 30 minutes. After that, 0.5 g solid sodium oleate was added.

3.3 ml of a particle dispersion according to example 5 (0.97 mol/l Fe) and 2.14 ml tetraethoxysilane were added to 120 ml of a mixture of water/ethanol (3:1) and 1.5 percent by weight ammonium. The dispersion was stirred during addition and after that treated with ultrasound for six hours. The dispersion was purified by centrifugation and redispersion in water.

### Example 2

### Sponge

Wound Insert Impregnated with Nanoparticles

A commercially available Tabotamp® sponge was dipped in the nanoparticle suspension produced according to example 1 for 6 minutes. The dipping process was repeated twice after drying. Alternatively, the suspension can be applied with a pipette. This process can be repeated several times until the desired loading of the sponge will be reached.

### Example 3

### Medical Cellulose

Medical Cellulose Coated with Nanoparticles

A piece of a wound cover, 3 cm wide and 6 cm long, such as SeaSorb from the company Coloplast, that includes calcium alginate and sodium carboxymethyl cellulose, was sprayed five times with ca. 1 ml of the nanoparticle suspension according to example 1 and was air-dried for ca. 20 minutes after each spraying step. Alternatively, the suspension can be applied with a pipette. This process can be repeated several times until the desired loading of the wound cover will be reached.

### Example 4

### Medical Cellulose with Drug

Medical Cellulose Impregnated with Nanoparticles and Cytostatic

Commercially available medical cellulose made of sodium carboxymethyl cellulose, poly-N-vinylpyrrolidone and polyethylene oxide (5 cm²) was dipped in a nanoparticle suspension produced according to example 1 for five minutes which contained a 0.3 mg/ml paclitaxel solution. The medical product will be ready-to-use after drying and sterilization.

### Example 5

### Gel

### Production of a Gel:

4 g of a mixture of collagen type I and collagen type II are dissolved in a liter of a 50 mM acetic acid solution. The collagen solution is centrifuged at 9,500 revolutions per minute at 4° C. for 45 minutes. The supernatant is decanted, filled into a dialysis tube and dialyzed against 25 liters of a 1 M acetic acid solution for two days and dialyzed thereafter against water for further four days.

After that, the collagen solution was concentrated within the dialysis tube to a concentration of 20 mg/ml (2% w/v).

For the production of the gel 10 ml of the collagen solution were incubated with 0.1 ml of a 1 N NaOH solution and 1 ml DMEM (Dulbecco's Modified Eagle Medium 10×) at 37° C. for one hour.

After that, 1.5 g of the lyophilized nanoparticles with a size distribution of 1-100 nm were added.

The gel was applied to the operative field as complete as possible after surgical removal of a solid small intestine tumor.

A successive treatment by thermotherapy in an alternating magnetic field showed a warming to 53° C. of the field of surgery.

### Example 6

### Gel with Drug

0.1 g of a cytostatic, temozolomide, are added to 10 g of the gel produced according to example 5 and mixed well after that.

Application of the gel occurred as described in example 5.

### Example 7

### Sponge

2 g of globin powder are produced as described in US 2007031474A.

A sponge-like implant is produced by lyophilizing a 1% aqueous suspension of oxidized cellulose in 1.5 percent by weight globin powder at a pH-value of 7.2. The oxidized cellulose can be used in the form of fibers or two- or three-dimensional structures, too.

The resulting sponge-like structure includes ca. 100 mg of oxidized cellulose and 40 to 200 mg globin and has a volume of ca. 10 cm³ and a thickness of ca. 3 mm.

The sponge-like structure is sterilized by ethylene oxide and packed.

### Example 8A

### Particle with Drug

Production of Nanoparticles with Attached Mitomycin:

To begin with, a conjugate of mitomycin and an aldehyde-functionalized alkoxysilane (e.g. triethoxysilylbutyl aldehyde) is synthesized for the coupling of the cytostatic mitomycin to aminosilane-stabilized iron oxide nanoparticles. That way the drug is coupled via an imine bonding. While stirring this conjugate is added to an aqueous dispersion of aminosilane-stabilized particles such as those from WO 97/38058 A. Ethylene glycol is added to the mixture and the water removed by distillation. As a result, the conjugate between drug and silane is coupled (condensed) to the shell already present on an aminosilane basis. Purification occurs by dialysis against ultrapure water. A detailed description of the reaction is included in WO 2006108405 A2.

### Example 8B

The production of nanoparticles with doxorubicin bound to the particle via an avidin bridge is performed as described in WO 2006108405 A2.

### Example 8C

The production of nanoparticles with doxorubicin coupled via a nucleotide sequence is performed as described in WO 2006108405 A2.

### Example 9

### Sponge

A sponge-like structure is produced as described in example 7, wherein instead of oxidized cellulose a mixture of collagen type I, collagen type II and chitosan (25:25:50 percents by weight) are used.

After that, the resulting sponge will be soaked with an aqueous suspension of nanoparticles coupled to doxorubicin according to example 8B or 8C and dried.

Instead of subsequent soaking the suspension according to example 7 can also be added to the nanoparticle suspension according to examples 8A, 8B or 8C and can be lyophilized together with the other components.

### Example 10

### Medical Cellulose

Medical cellulose on the basis of chitosan, uronic acid and carboxymethyl dextrane (4 cm², ca. 20 mg) is spread flatly in a culture dish and is being dripped onto with an aqueous suspension containing the nanoparticles with coupled mitomycin according to example 8A until the loading of the cellulose is achieved with 50 mg of nanoparticles.

### Example 11

### Gel with Nanoparticles

23.5 percent of weight of non-hydrated lecithin, 20.0 percent of weight of propylene glycol, 10.0 percent of weight of ethanol, 2.5 percent of weight of sorbitol, 0.05 M phosphate buffer (ad 100.0%) were stirred at room temperature for 16 hours.

The such resulting gel was stirred together with the nanoparticle suspension of example 1 for 4 h to obtain a nanoparticle gel.

### Example 12

### Film-Forming Spray with Nanoparticles

172 g maleic acid diethyl ester (afflux 1), 98 g maleic acid anhydride (afflux 2, in a heatable dropping funnel), 200 g vinyl isobutyl ether (afflux 3) and 12 g tert.-butyl perneodecanoate (afflux 4) are filled into the corresponding metering vessels. First, 111 ml of afflux 1, 10 ml of afflux 3 and 3 ml of afflux 4 are put in a 2 1-agitator vessel which is equipped with a stirrer, heating, reflux condenser and prepared dosing devices as well as inlet and outlet for gas and are heated to 60° C. Residual afflux 1, residual afflux 3 and afflux 2 are additively dosed within 3 hours and residual afflux 4 within 4 hours at this temperature. Afterwards it is stirred yet for 1 hour at 80° C. A colorless highly viscous melt is obtained which is mixed with 18 g of water at this temperature and stirred for 1 h. After cooling to 75° C., 480 g of ethanol are additively dosed within 15 minutes and stirred for 1 h at this temperature. After cooling to 25° C. a clear viscous polymer solution with a solid content of 48.1 percent by weight results.

The such obtained viscous polymer solution was stirred with the nanoparticle suspension of example 1 for 2 h to obtain a film-forming nanoparticle spray.

### Example 13

### Film-Forming Spray with Nanoparticles and Drug

10 ml of the polymer solution obtained according to example 12 are mixed with 100 mg carboplatin and 1000 mg lyophilized nanoparticles according to example 1.

### Example 14

### Treatment of Cervix, Thoracic Wall and ENT Tumors

A carrier material soaked with a nanoparticle solution according to example 1A (2 molar & 3 molar) is applied to a bone. The bone is positioned in the therapeutic device and exposed to an alternating magnetic field. The increase in temperature to be measured on top of the bone is determined at an ambient temperature as constant as possible. This experimental set-up illustrates that cervix, thoracic wall and ENT tumors can be treated in an alternating magnetic field by means of nanoparticle-coated carriers which are applied onto a bone or in the vicinity of a bone.

### Materials

Equipment:
   Therapeutic device MFH-12TS,
   Recirculating cooler (Julabo; FC600S) with tube connections,
   Fixator (rats) with tube connections,
   Polytec Luxtron (model: LAB. KIT) with 2 temperature measuring sensors,
   Measuring device for field strength (with sensor),
   Water bathes (37° C.),
   Calibration sensor (calibrated until 11/09)
Material:
   2 M or 3 M nanoparticle suspension according to example 1A: sonicated for 15 minutes each
   Carrier material:
      1: SPONGOSTAN powder
         (1 g, resorbable gelatine powder, haemostatic; Johnson+Johnson)
      2: SPONGOSTAN Special
         (7×5×0.1 cm, resorbable haemostatic gelatine sponge; Johnson & Johnson)
      3: Gelita tampon
         (1×1×1 cm, sponge-like, made of hardened gelatine of porcine origin, biodegradable anti-haemorrhagic agent; B. Braun Melsungen AG)
      4: Lyostypt®
         (3×5 cm, wet-stable compress of native collagen of bovine origin for localized hemostasis, resorbable; B. Braun Melsungen AG)
   Bones (“porcine spare ribs”),
   Forceps,
   Plasticine mass,
   Medical plaster, (Durapore™; 3 M; 2.5 cm×9.14 m)
   Cold/Warm compress (Pharma-Depot GmbH; 13×14 cm)
   Tuberculin syringe (Omnifix®-F; Braun; 0.01 mL/1 mL),
   Disposable injection cannula (Sterican®; Braun; 27G×11/2″, 0.40×40 mm),
   Vernier Caliper (DialMax. calibrated until 08/09; MS150-4/Atl),
   Scalpel (blade no. 11),
   Beakers,
   Camera,
Chemicals:
   Hydrogen peroxide (H₂O₂; 30%),
   Alginate (alginic acid sodium salt),

### Experimental Set-Up

A suitable fixator was tempered to 55° C. by a recirculating cooler. Tightness tests were performed.
1. The fixator was positioned in the slot of the therapeutic device,
2. a pre-warmed (37° C.) cold/warm compress was put into the “head area” of the fixator (reduced the air volume inside the device and slightly “buffers” the temperature oscillations slightly),
3. bone:
   A bone was separated from the spareribs and was roughly ridded of meat,
   Put into a beaker of H₂O₂,
   Subsequently the bone was “cleansed” with a scalpel,
   The bone was separated into experimentally usable parts with a saw.
4. Field strength:
   The measuring head for the measurement of the field strength is positioned at that position in the fixator where the measurements will be performed later, plasticine serves as marking aids herein
   3 Field strengths (relevant for clinical application) will be analyzed: 3.0 kA/m, 3.5 kA/m, 4.0 kA/m
   These measurements yielded the following values:

| kA/m | % field strength |
|---|---|
| 3.03 | 14.5 |
| 3.49 | 17.0 |
| 4.07 | 20.5 |

% field strength is that device setting which corresponds to the assigned field strength in kA/m

5. the temperatures of the air of the internal space and of the applicator bottom are determined in the fixator.

### Example 14A

### Lyostypt®

Particle: Nanoparticle suspension according to example 1A (0.5 mL, 2 molar)
Carrier: Lyostypt®, size: (19.95×14.9×3.4) mm
Bone: size: (44.4×13.2×10.9) mm

A bone fragment was measured [measurements: (44.4×13.2×10.9) mm] and part of the carrier was cut to size [measurements: (19.95×14.9×3.4) mm]. The carrier is positioned on the bone and soaked with the particles (0.5 mL, 2 molar according to example 1A). The loaded bone is positioned in the applicator [sensor 1 (red): perpendicular from above onto the soaked carrier; sensor 2 (blue): base value (“empty” bone)], and values for the carrier are determined:

Field strength is to be: 3.0 kA/m → 14%

| | |
|---|---|
| 0:00:00 | Sensor 1: ca. 33° C., MF↑, fan↑ |
| 2:00 | Sensor 1: ca. 36.5° C. |
| 5:15 | Sensor 1: ca. 36.0° C., carrier substance is slowly slipping |
| | away from under the sensor |
| 8:40 | Sensor 1: ca. 35.9° C. |
| 9:40 | Sensor 1↓ because “pulled out” |
| 10:00 | Sensor 1: ca. 35.8° C., MF↓ |
| 11:48 | Sensor 1: 32.8° C., probe re-adjusted |
| 12:30 | Sensor 1: 32.5° C., MF↑ (14%) |
| 17:26 | Sensor 1: 35.4° C., MF↓ |
| 20:44 | New position: Sensor lies between carrier substance and bone |
| 21:45 | Sensor 1: 31.8° C., MF↑ (14%), fan↑ |
| 26:47 | Sensor 1: 33.7° C., MF↓ |
| 28:30 | fan↓ |

Field strength is to be: 3.5 kA/m → 17%
(new piece of carrier substance with identical measures and
volume Nanotherm, sensor between carrier substance and bone)

| | |
|---|---|
| 0:00:00 | Sensor 1: 26.0° C. MF↑. Fan↑ |
| 2:00 | Sensor 1: 30.3° C. |
| 2:35 | Sensor 1: 31.0° C. |
| 2:58 | Sensor 1: 31.3° C. |
| 3:26 | Sensor 1: 31.6° C. |
| 3:46 | Sensor 1: 31.9° C. |
| 4:00 | Sensor 1: 32.0° C. |
| 5:00 | Sensor 1: 32.5° C. |
| 5:44 | Sensor 1: 32.8° C. |
| 6:51 | Sensor 1: 33.0° C. |
| 8:00 | Sensor 1: 33.4° C. |
| 9:00 | Sensor 1: 33.5° C. |
| 10:00 | Sensor 1: 33.6° C. MF↓ |
| ca. 12:30 | Sensor 1: 29.0° C. |
| ca. 14:00 | Sensor 1: 29.2° C. |

Field strength is to be: 4.0 kA/m → 20.5%
(unmodified set-up)

| | |
|---|---|
| 0:00:00 | Sensor 1: 29.3° C. MF↑. Fan↑ |
| 1:00 | Sensor 1: 33.0° C. |
| 2:00 | Sensor 1: 34.7° C. (Ambient air draught! Cause?) |
| 3:15 | Sensor 1: 35.4° C. |
| 4:00 | Sensor 1: 35.7° C. |
| 5:15 | Sensor 1: 35.9° C. |
| 6:30 | Sensor 1: 36.0° C. |
| 8:00 | Sensor 1: 36.0° C. |
| 9:00 | Sensor 1: 36.1° C. |
| 9:15 | Sensor 1: 36.0° C.; Sensor 2: 34.5° C.; MF↓ |
| ca. 12:00 | Sensor 1: 30.5° C.; Sensor 2: 34.6° C.; Fan↓ |

The sensor is always positioned between the carrier and the bone.

MF↑: Alternating magnetic field on
MF↓: Alternating magnetic field off

### Fan↑: Fan on

### Fan↓: Fan off

Sensor 1↓: Sensor does not function

### Example 14B

### SPONGOSTAN

Particle: Nanoparticle suspension according to example 1A (1.5 mL, 2 molar)
Carrier: Spongostan powder, mass: 0.3 g
Bone: size: (44.4×13.2×10.9) mm

1.08 g powder (carrier) is soaked in 1.5 mL of particles (2 molar according to example 1A) and mixed well, and a partial quantity of m=0.46 g of the soaked carrier is modelled on the bone.

The measurement head for the field strength measurements is positioned at that position at the fixator where the measurements will be performed later; plasticine serves as marking aids. Three field strengths (relevant for clinical application) are analyzed: 3.0 kA/m, 3.5 kA/m, 4.0 kA/m

Field strength is to be: 3.0 kA/m → 14%

| | |
|---|---|
| 0:00:00 | Sensor 1: 33.6° C., MF↑, fan↑ |
| 2:00 | Sensor 1: 34.8° C. |
| 10:00 | Sensor 1: 35.7° C., MF↓ |
| after 2′ | Sensor 1: 35.3° C. |

Field strength is to be: 3.5 kA/m → 17%

| | |
|---|---|
| 0:00:00 | Sensor 1: 35.8° C., MF↑ (fan on) |
| 2:00 | Sensor 1: 36.0° C. |
| 4:00 | Sensor 1: 36.1° C. |
| 5:00 | Sensor 1: 36.3° C., MF↓ |

Field strength is to be: 4.0 kA/m → 20.05%

| | |
|---|---|
| 0:00:00 | Sensor 1: 36.8° C., MF↑, fan↑ |
| 7:50 | Sensor 1: 39.0° C. |
| 9:00 | Sensor 1: 39.2° C. |
| 10:00 | Sensor 1: 39.3° C., MF↓ |
| after 2′ | Sensor 1: 38.8° C. |

### Example 14C

### SPONGOSTAN

Particle: Nanoparticle suspension according to example 1A (1.6 mL, 2 molar)
Carrier: Carrier soaked according to example 14B, mass: ca. 0.8 g

### Bone: Size: (44.4×13.2×10.9) mm

The remaining amount of ca. 0.8 grams of soaked carrier from example 14B is mixed with 1.6 ml particles (2 molar according to example 1A) and is applied on the bone cleansed according to example 14. Again the sensor is positioned between bone and carrier. It was measured again at the 3 field strengths (3.0 kA/m, 3.5 kA/m, 4.0 kA/m).

Field strength is to be: 3.0 kA/m → 14%

| | |
|---|---|
| 0:00:00 | Sensor 1: 24.1° C., MF↑, fan↑ |
| 7:00 | Sensor 1: 32.0° C. |
| 10:00 | Sensor 1: 33.2° C. |
| 12:02 | Sensor 1: 33.5° C. MF↓ |
| after 4′ | Sensor 1: 30.8° C. |

Field strength is to be: 3.5 kA/m → 17%

| | |
|---|---|
| 0:00:00 | Sensor 1: 30.7° C., MF↑ (fan on) |
| 1:00 | Sensor 1: 33.1° C. |
| 5:30 | Sensor 1: 35.6° C. |
| 8:00 | Sensor 1: 35.7° C. |
| 10:00 | Sensor 1: 36.1° C., MF↓ |
| after 1′ | Sensor 1: 33.3° C. |
| after 2′ | Sensor 1: 32.2° C. |
| after 3′ | Sensor 1: 31.8° C. |

Field strength is to be: 4.0 kA/m → 20.05%

| | |
|---|---|
| 0:00:00 | Sensor 1: 31.6,8° C., MF↑, fan↑ |
| 1:00 | Sensor 1: 34.7° C. |
| 4:00 | Sensor 1: 37.0° C. |
| 5:00 | Sensor 1: 37.4° C. |
| 6:00 | Sensor 1: 37.7° C. |
| 7:00 | Sensor 1: 37.6° C. |
| 9:00 | Sensor 1: 37.9° C. |
| 10:00 | Sensor 1: 38.2° C., MF↓ |
| after 30″ | Sensor 1: 35.4° C. |

### Example 14D

### SPONGOSTAN Special

Particle: Nanoparticle suspension according to example 1A (1.0 mL, 2 molar)
Carrier: Carrier soaked according to example 14B, size: (10.0×10.0×2.0) mm

### Bone: Size: (44.4×13.2×10.9) mm

The carrier must be soaked with the particle suspension (15 minutes) in order that the carrier absorbs the particles (1.0 mL, 2 molar according to example 1A). 1 mL of particle suspension is injected to the carrier into a packaging [m=0.00]; the ashlar absorbs a maximum and is put onto the bone.

Measurements are performed as described in example 14A.

Field strength is to be: 3.0 kA/m → 14%

| | |
|---|---|
| 0:00:00 | Sensor 1: 25.5° C., MF↑, fan↑ |
| 6:30 | Sensor 1: 30.6° C. |
| 8:00 | Sensor 1: 31.1° C. |
| 9:00 | Sensor 1: 31.4° C. |
| 10:00 | Sensor 1: 31.7° C. |
| 11:00 | Sensor 1: 32.0° C. |
| 12:00 | Sensor 1: 32.0° C. |
| 12:30 | Sensor 1: 32.0° C., MF↓ |
| after 1′30″ | Sensor 1: 29.8° C., fan↓ |
| after 3′ | Sensor 1: 29.6° C. |

Field strength is to be: 3.5 kA/m → 17%

| | |
|---|---|
| 0:00:00 | Sensor 1: 29.6° C., MF↑, fan↑ |
| 1:00 | Sensor 1: 32.0° C. |
| 5:00 | Sensor 1: 34.1° C. |
| 10:00 | Sensor 1: 34.6° C., MF↓ |
| after 1′ | Sensor 1: 31.8° C. |

Field strength is to be: 4.0 kA/m → 20.05%

| | |
|---|---|
| 0:00:00 | Sensor 1: 30.5° C., MF↑, fan↑ |
| 2:30 | Sensor 1: 35.5° C. |
| 4:00 | Sensor 1: 36.2° C. |
| 5:00 | Sensor 1: 36.6° C. |
| 10:00 | Sensor 1: 36.8° C., MF↓ |

### Example 14E

### Gelita Tampon

Particle: Nanoparticle suspension according to example 1A (1.0 mL, 2 molar)
Carrier: Gelita tampon, size: (1×1×1) cm
Bone: size: (44.4×13.2×10.9) mm

The carrier must be soaked with the particle suspension (15 minutes) in order for the carrier to absorb the nanoparticle suspension (1.0 mL, 2 molar according to example 1a). 1 mL of particle suspension is injected into the packaging to the carrier Gelita tampon; the cube absorbs a maximum (Gelita tampon [m=0.00] with nanoparticle suspension: m=0.45 g) and is put onto the bone.

Field strength is to be: 3.0 kA/m → 14%

| | |
|---|---|
| 0:00:00 | Sensor 1: 29.1° C., MF↑, fan↑ |
| 5:00 | Sensor 1: 34.7° C. |
| 7:00 | Sensor 1: 35.3° C. |
| 10:00 | Sensor 1: 35.8° C. |

Field strength is to be: 3.5 kA/m → 17%

| | |
|---|---|
| 0:00:00 | Sensor 1: 33.7° C., MF↑, (fan on) |
| 1:00 | Sensor 1: 35.2° C. |
| 5:00 | Sensor 1: 36.7° C. |
| 10:00 | Sensor 1: 37.1° C., MF↓ |

Field strength is to be: 4.0 kA/m → 20.,05%

| | |
|---|---|
| 0:00:00 | Sensor 1: 34.9° C., MF↑, (fan on) |
| 2:00 | Sensor 1: 37.8° C. |
| 3:00 | Sensor 1: 38.3° C. |
| 5:00 | Sensor 1: 38.8° C. |
| 7:00 | Sensor 1: 39.0° C. |
| 10:00 | Sensor 1: 39.2° C., MF↓ |

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A solid or gel-like medical product heatable by an alternating magnetic field, wherein the medical product is provided in the form of a physiologically acceptable tissue, sponge, film or gel and wherein magnetic particles are contained in the medical product which will generate heat if excited by an alternating magnetic field and thereby will heat the medical product.

2. Medical product according to claim 1, wherein the particles are stationary embedded into or attached to the medical product.

3. Medical product according to claim 1, wherein the particles will remain permanently in the medical product, will not be released by diffusion and will only be released in the case of biodegradable medical products due to the degradation process.

4. Medical product according to claim 1, wherein the medical product is deformable and can be adjusted to the surface contours of a tissue or organ or the field of surgery after surgical tumor removal.

5. Medical product according to claim 1, wherein the medical product is biodegradable.

6. Medical product according to claim 1, wherein the particles are microparticles or nanoparticles.

7. Medical product according to claim 1, wherein the particles are paramagnetic or superparamagnetic.

8. Medical product according to claim 1, wherein the medical product is impregnated, coated or soaked with the magnetic particles.

9. Medical product according to claim 1, wherein the medical product is bioresorbable within one to twelve months.

10. Medical product according to claim 1, wherein the medical product is physiologically tolerated and is degraded to physiologically tolerated components.

11. Medical product according to claim 1, wherein the medical product is flexible and does not consist of a metal or a metal alloy and wherein the medical product is not provided in form of an aqueous solution of the particles.

12. Medical product according to claim 5, wherein the biodegradable medical product releases the magnetic particles and/or delivers them to surrounding tumor tissue or tumor cells.

13. Medical product according to claim 1, wherein the magnetic particles are provided at concentrations of 10 to 100 milligrams per square centimeter surface of the medical product.

14. Medical product according to claim 1, wherein the magnetic particles are provided at concentrations of 100 micrograms to 2 grams per gram of the medical product.

15. Medical product according to claim 1, wherein the medical product is medical cellulose, bandaging materials, wound inserts, surgical sutures, compresses, sponges, medical textiles, ointments, gels, film-forming compositions or film-forming sprays.

16. Medical product according to claim 1, further comprising at least one therapeutic effective agent selected from to group comprising anti-proliferative, anti-migratory, anti-angiogenic, anti-thrombotic, anti-inflammatory, anti-phlogistic, cytostatic, cytotoxic, anti-coagulative, anti-bacterial, anti-viral and/or anti-mycotic drugs, in particular wherein the at least one therapeutic active substance is selected from the group comprising actinomycin S, aminoglutethimide, arnsacrin, anastrozol, antagonists of purine or pyrimidine bases, anthracycline, aromatase inhibitors, asparaginase, anti-estrogens, bexaroten, bleomycin, buselerin, busulfan, camptothecin derivatives, capecitabin, carboplatin, carmustin, chlorambucil, cisplatin, cladribine, cyclophosphamide, cytarabine, cytosine arabinoside, alkylating cytostatics, dacarbazine, dactinomycin, daunorubicin, docetaxel, doxorubicin, epirubicin, estramustin, etoposide, exemestan, fludarabine, fluorouracil, folic acid antagonists, formestan, gemcitabine, glucocorticoids, goselerin, hormones and hormones antagonists, hycamtin, hydroxylurea, idarubicin, ifosfamide, imatinib, irinotecan, letrozol, leuprorelin, lomustin, melphalan, mercaptopurine, methotrexate, miltelosiii, mitomycin, mitosis inhibitors, mitoxantrone, nimustine, oxaliplatin, paclitaxel, pentostatin, procarbazine, tamoxifen, temozolomide, teniposide, testolacton, thiotepa, tioguanine, topoisomerase inhibitors, topotecan, treosulfan, tretinoin, triptorelin, trofosfamide, vinblastin, vincristin, vindesin, vinorelbin, cytostatically effective antibiotics.

17. (canceled)

18. Medical product according to claim 16, wherein the at least one drug is bound adhesively, ionically, covalently or via a linker to the particle.

19. Medical product according to claim 16, wherein the detachment of the at least one drug from the carrier is initiated by an alternating magnetic field.

20. Medical product according to claim 1, wherein the medical product consists of the following materials: Polyacrylic acid, polyacrylate, polymethyl methacrylate, polybutyl methacrylate, polyisobutyl methacrylate, polyacrylamide, polyaerylnitrile, polyamide, polyetheramidc, polyethyleneamine, polyimide, polycarbonate, polycarbourethane, polyvinylketone, polyvinylhalogenide, polyvinylidenhalogenide, polyvinylether, polyvinyl aromatics, polyvinyl ester, polyvinylpyrollidone, polyoxymethylene, polyethylene, polypropylene, polytetrafluoroethylene, polyurethane, polyolefin elastomer, polyisobutylene, EPDM gums, fluorosilicone, carboxymethylchitosan, polyethylenetcrephtalate, polyvalerate, carboxymethylcellulose, cellulose, rayon, rayon triacetate, cellulose nitrate, cellulose acetate, hydroxyethyl cellulose, cellulose butyrate, cellulose acetate-butyrate, ethylvinylacetate copolymer, polysulfonc, polyethersulfone, epoxy resins, ABS resins, EPDM gums, silicone pre-polymer, silicone, polysiloxanc, polyvinyl halogen, cellulose ether, cellulose triacetate, chitosan, chitosan derivatives, polymerizable oils, polyvalerolactone, poly-ε-decalacton, polylactide, polyglycolide, co-polymers of polylactide and polyglycolide, poly-ε-caprolactone, polyhydroxy buturic acid, polyhydroxybutyrate, polyhydroxyvalerate, polyhydroxybutyrate-co-valerate, poly(1,4-dioxan-2,3-dione), poly(1,3-dioxan-2-one), polypara-dioxanonc, polyanhydridc, polymalcic acid anhydride, polyhydroxy methacrylate, polycyanoacrylate, polycaprolacton dimethylacrylate, poly-β-maleic acid, polycaprolactonbutyl acrylate, multi-block polymers made of oligocaprolactonediol and oligodioxanondiol, polyetherester-multi-block polymers made of PEG and poly(butyleneterephtalate), polypivotolactone, polyglycolic acid trimethylcarbonate, polycaprolactone-glycolide, poly(γ-ethylglutamate), poly(DTH-iminocarbonate), poly(DTE-co-DT-carbonate), poly(bisphenol A-iminocarbonate), polyorthoester, polyglycolic acid trimethyl-carbonate, polytrimethylcarbonate, polyimino carbonate, polyvinylic alcohols, polyester amides, glycolidized polyesters, polyphosphocsters, polyphosphazenes, poly[p-carboxyphenoxy)propane], polyhydroxypentaic acid, polyethylene oxide-propylene oxide, soft polyurethanes, polyurethanes with amino acid rests in the backbone, polyether esters, polyethylene oxide, polyalkenoxalates, polyorthoesters, carrageenans, starch, collagen, protein-based polymers, polyamino acids, synthetic polyamino acids, zein, modified zein, polyhydroxyalkanoates, pectic acid, actinic acid, fibrin, modified fibrin, casein, modified casein, carboxymethylsulphate, albumin, hyaluronic acid, heparan sulphate, heparin, chondroitin sulphate, dextrane, cyclodextrine, co-polymers made of PEG and polypropyleneglycol, gam arabic, guar, or other gum resins, gelatine, collagen, collagen-n-hydroxysuccinimide, lipids, lipoids, polymerizable oils and their modifications, co-polymers and mixtures of the aforementioned substancess.

21. Medical product according to claim 1 for use in medicine.

22. Use of the medical product according to claim 1 for the production of a means for prophylaxis, treatment and after-treatment of cancer, tumors and proliferative diseases, in particular for the after-treatment of an operative field in a cancer surgery.

23. (canceled)

24. Use of the medical product according to claim 22, wherein cancer, tumor or proliferative disease are selected from the group comprising: Adenocarcinomas, choroid melanoma, acute leukemia, acoustic neurinoma, ampullary carcinoma, anal carcinoma, astrocytomas, basalioma, pancreatic carcinoma, connective tissue tumor, bladder cancer, bronchial carcinoma, non-small cell bronchial carcinoma, breast cancer, Burkitt's lymphoma, corpus carcinoma, CUP syndrome, colon cancer, cancer of the small intestine, small intestine tumors, ovarian cancer, endometrial carcinoma, ependymoma, epithelial cancers, Ewing's sarcoma, gastrointestinal tumors, gall-bladder cancer, bilary carcinomas, uterine cancer, cervical cancer, glioblastomas, gynecological tumors, otorhinolaryngologic tumors, hematologic neoplasias, urethral cancer, skin cancer, brain tumors (gliomas), brain metastases, testicular cancer, hypophyseal tumor, carcinoids, Kaposi's sarcoma, laryngeal cancer, germinal tumor, bone cancer, colorectal carcinoma, head-neck tumors (tumors of neck, nose and ear areas), colon carcinoma, craniopharyngiomas, cancer of the mouth area and the lips, hepatic cancer, hepatic metastases, eyelid tumor, lung cancer, lymphatic gland cancer (Hodgkin/Non-Hodgkin), lymphomas, stomach cancer, malignant melanoma, malignant neoplasia, malignomas of the gastrointestinal tract, mammary carcinoma, rectal cancer, medulloblastomas, melanoma, meningeomas, Hodgkin's disease, mycosis fungoides, nose cancer, neurinoma, neuroblastoma, kidney cancer, renal cell carcinoma, Non-Hodgkin's lymphomas, oligodendroglioma, esophageal carcinoma, osteolytic carcinoma and osteoplastic carcinoma, osteosarcoma, ovarian carcinoma, pancreatic carcinoma, penile cancer, squamous cell carcinomas of the head and neck, prostate cancer, pharyngeal cancer, rectal carcinoma, retinoblastoma, vaginal cancer, thyroid carcinoma, Schneeberger's disease, esophageal cancer, spinalioma, T-cell lymphoma (mycosis fungoides), thymoma, tubal carcinoma, eye tumors, urethral cancer, urologic tumors, urothelial carcinoma, vulvar cancer, mastoid involvement, soft tissue tumors, soft tissue sarcoma, Wilms' tumor, cervix carcinoma and tongue cancer.
